(19) United States
(12) Patent Application Publication
(10) Pub. No.: US 2011/0014291 A1
(43) Pub. Date: Jan. 20, 2011

(54) Novel Polymorphs of Bosentan

(75) Inventors: Girish Dixit, Ghaziabad (IN); Nandkumar Gaikwad, Maharashtra (IN); Hima Prasad Naidu, Andhra Pradesh (IN); Nitin Sharadchandra Pradhan, Maharashtra (IN); Jon Valgeirsson, Hafnarfjordur (US)

Correspondence Address: CANTOR COLBURN LLP, 20 Church Street, 22nd Floor, Hartford, CT, 06103, US

(73) Assignee: ACTAVIS GROUP PTC EHF, 220 Hafnarfjordur (IS)

(21) Appl. No.: 12/682,112

(22) PCT Filed: Oct. 13, 2008
(86) PCT No.: PCT/IB2008/003262

§ 371 Date: Oct. 4, 2010

(30) Foreign Application Priority Data
Oct. 11, 2007 (IN) 2289/CHE/2007

(51) Int. Cl. A61K 9/14; C07D 403/04; A61K 31/506; A61P 9/10; A61P 9/12

(52) U.S. Cl. 424/489; 544/296; 514/269

(57) ABSTRACT

Disclosed herein are novel polymorphic forms of bosentan, processes for preparation, pharmaceutical compositions, and method of treating thereof.

### CROSS REFERENCE TO RELATED APPLICATION

This application claims the benefit of priority to Indian provisional application No. 2289/CHE/2007, filed on Oct. 11, 2007, which is incorporated herein by reference.

### FIELD OF THE INVENTION

The present invention provides novel polymorphic forms of bosentan, process for preparation, pharmaceutical compositions, and method of treating thereof.

### BACKGROUND OF THE INVENTION

U.S. Pat. No. 5,292,740 discloses a variety of sulfonamide derivatives, processes for the preparation, pharmaceutical compositions and method of use thereof. These compounds are useful in treatment of a variety of illness including cardiovascular disorders such as hypertension, ischemic, vasospasms and angina pectoris. Among them, Bosentan, p-tert-butyl-N-[6-(2-hydroxyethoxy)-5-(2-methoxyphenoxy)-2-(2-pyrimidinyl)-4-pyrimidinyl]benzenesulfonamide monohydrate, has a wide variety of biological activities including inhibiting the renin angiotensin system and acting as an endothelin antagonist. Bosentan blocks the binding of endothelin to its receptors, thereby negating endothelin's deleterious effects. Bosentan has the molecular formula of C₂₇H₂₉N₅O₆S.H₂O, molecular weight of 569.63 and a structural formula of:

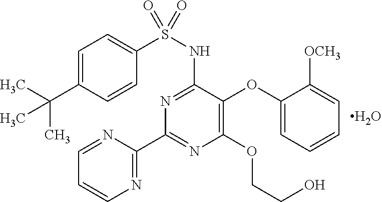

Various processes for the preparation of Bosentan and related compounds were disclosed in U.S. Pat. No. 5,292,740 and U.S. Pat. No. 6,136,971.

According to the U.S. Pat. No. 5,292,740 (hereinafter referred to as the '740 patent), Bosentan can be prepared by the reaction of 5-(2-methoxyphenoxy)-2-(2-pyrimidin-2-yl)-4,6(1H,5H)-pyrimidinedione with phosphorous oxychloride in acetonitrile to give 4,6-dichloro-5-(2-methoxyphenoxy)-2,2′-bipyrimidine, which by condensation with 4-tert-butylbenzenesulfonamide potassium in dimethylsulfoxide followed by treatment with hydrochloric acid to afford p-tert-butyl-N-[6-chloro-5-(2-methoxyphenoxy)-2-(2-pyrimidinyl)-4-pyrimidinyl]benzenesulfonamide, which is then reacted with a sodium ethylene glycol in ethylene glycol solvent to produce bosentan as sodium salt (m.p. 195-198° C.). According to the U.S. Pat. No. 6,136,971 (hereinafter referred to as the '971 patent), Bosentan can be prepared by the reaction of 5-(2-methoxyphenoxy)-2-(2-pyrimidin-2-yl)-4,6(1H, 5H)-pyrimidinedione with phosphorous oxychloride in toluene to give 4,6-dichloro-5-(2-methoxyphenoxy)-2,2′-bipyrimidine, which by condensation with 4-tert-butylbenzenesulfonamide in the presence of anhydrous potassium carbonate and a phase transfer catalyst (e.g., benzyltriethylammonium chloride) in toluene to afford p-tert-butyl-N-[6-chloro-5-(2-methoxyphenoxy)-2-(2-pyrimidinyl)-4-pyrimidinyl]benzenesulfonamide potassium salt, which is then reacted with ethylene glycol mono-tert-butyl ether in toluene in the presence of granular sodium hydroxide to give p-tert-butyl-N-[6-(2-tert-butyl-ethoxy)-5-(2-methoxyphenoxy)[2,2′-bipyrimidin]-4-yl]benzene-sulfonamide (Bosentan tert-butyl ether). The Bosentan tert-butyl ether obtained is then reacted with formic acid followed by treatment with absolute ethanol to afford bosentan formate monoethanolate, which by reaction with sodium hydroxide in absolute ethanol and water followed by acidification with hydrochloric acid and then the resulting precipitate is suction-filtered, washed with ethanol-water mixture (1:1) to give Bosentan crude. The crude Bosentan obtained is then purified with mixture of ethanol and water and the resulting precipitate is suction-filtered to give bosentan. The '971 patent makes no reference to the existence of specific polymorphic forms of bosentan.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

It has been disclosed in the art that the amorphous forms in a number of drugs exhibit different dissolution characteristics and in some cases different bioavailability patterns compared to crystalline forms [Konne T., Chem. Pharm. Bull., 38, 2003-2007 (1990)]. For some therapeutic indications one bioavailability pattern may be favored over another.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Hence, there is a need in the art for novel and stable polymorphic forms of bosentan.

We have now surprisingly and unexpectedly discovered novel polymorphic forms of bosentan, different from the material obtained according to the teachings of the '971 patent, and having adequate stability and good dissolution properties.

In our hands, the methods of the '971 patent yield a crystalline form, which we denote as Form I, characterized by an X-ray powder diffraction pattern having peaks expressed as 2-theta angle positions at about 6.34, 10.77, 12.69, 15.85, 19.05, 19.84 and 21.29±0.2 degrees substantially as depicted in FIG. 9, different from the crystal forms of the present invention.

### SUMMARY OF THE INVENTION

In one aspect, the present invention provides a novel and stable crystalline form of bosentan, designated as bosentan crystalline Form A1, characterized by an X-ray powder diffraction pattern having peaks expressed as 2-theta angle positions at about 9.62, 16.34, 18.18 and 22.08±0.2 degrees.

In another aspect, the present invention further encompasses a process for preparing the highly pure and stable crystalline Form A1 of bosentan.

In another aspect, the present invention provides a novel and stable crystalline form of bosentan, designated as bosentan crystalline Form A2, characterized by an X-ray powder diffraction pattern having peaks expressed as 2-theta angle positions at about 8.26, 9.15, 15.21, 15.42, 16.63, 18.55 and 30.39±0.2 degrees.

In another aspect, the present invention further encompasses a process for preparing the highly pure and stable crystalline Form A2 of bosentan.

In another aspect, the present invention provides a novel and stable crystalline form of bosentan, designated as bosentan crystalline Form A4, characterized by an X-ray powder diffraction pattern having peaks expressed as 2-theta angle positions at about 4.04, 5.62, 7.84 and 17.06±0.2 degrees.

In another aspect, the present invention further encompasses a process for preparing the highly pure and stable crystalline Form A4 of bosentan.

In another aspect, the present invention provides a novel and stable amorphous form of bosentan.

In another aspect, the present invention further encompasses a process for preparing the highly pure and stable amorphous form of bosentan.

In another aspect, the present invention provides pharmaceutical compositions comprising a therapeutically effective amount of any one of the bosentan polymorphic forms or mixtures thereof of the present invention, and one or more pharmaceutically acceptable excipients.

In another aspect, the present invention provides pharmaceutical compositions comprising the polymorphic forms of bosentan prepared according to the processes of the present invention in any of its embodiments and one or more pharmaceutically acceptable excipients.

In yet another aspect, the present invention further encompasses a process for preparing a pharmaceutical formulation comprising combining any one of the polymorphic forms of bosentan prepared according to processes of the present invention in any of its embodiments, with one or more pharmaceutically acceptable excipients.

In another aspect, the substantially pure polymorphic forms of bosentan disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

Unless otherwise indicated, the following definitions are set forth to illustrate and define the meaning and scope of the various terms used to describe the invention herein.

The term “crystalline polymorph” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of bosentan in accordance with the present invention preferably contains less than about 10% crystalline forms of bosentan, more preferably less than 5% crystalline forms of bosentan, and still more preferably is essentially free of crystalline forms of bosentan. “Essentially free of crystalline forms of bosentan” means that no crystalline polymorph forms of bosentan can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use. Representative alkali or alkaline earth metal salts include the sodium, calcium, potassium and magnesium salts, and the like.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

As used herein, Dₓ means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “water content” refers to the content of water based upon the Loss on Drying method as described in Pharmacopeial Forum, Vol. 24, No. 1, page 5438 (January-February 1998), the Karl Fisher assay for determining water content or thermogravimetric analysis (TGA). The calculation of water content is based upon the percent of weight that is lost by drying.

By “substantially pure” is meant having purity greater than about 98%, specifically greater than about 99%, more specifically greater than about 99.5%, and most specifically greater than about 99.9% measured by HPLC.

### BRIEF DESCRIPTION OF THE DRAWINGS

FIG. 1 is a characteristic powder X-ray diffraction (XRD) pattern of bosentan crystalline Form A1.

FIG. 2 is a characteristic infra red (IR) spectrum of bosentan crystalline Form A1.

FIG. 3 is a characteristic powder X-ray diffraction (XRD) pattern of bosentan crystalline Form A2.

FIG. 4 is a characteristic infra red (IR) spectrum of bosentan crystalline Form A2.

FIG. 5 is a characteristic powder X-ray diffraction (XRD) pattern of bosentan crystalline Form A4.

FIG. 6 is a characteristic infra red (IR) spectrum of bosentan crystalline Form A4.

FIG. 7 is a characteristic powder X-ray diffraction (XRD) pattern of amorphous bosentan.

FIG. 8 is a characteristic infra red (IR) spectrum of amorphous bosentan.

FIG. 9 is a characteristic powder X-ray diffraction (XRD) pattern of bosentan crystalline Form I obtained as per the process exemplified in the '971 patent.

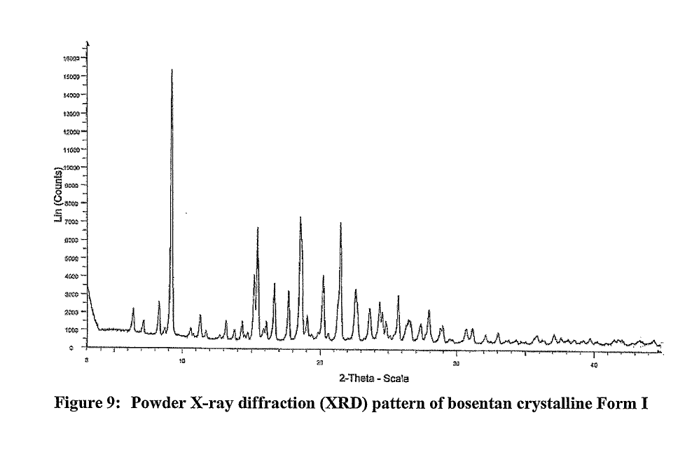

The X-Ray powder diffraction was measured by an X-ray powder diffractometer equipped with a Cu-anode (?=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2?; step width=0.01579°; and measuring time per step=0.11 second.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 or 450 cm⁻¹.

### DETAILED DESCRIPTION OF THE INVENTION

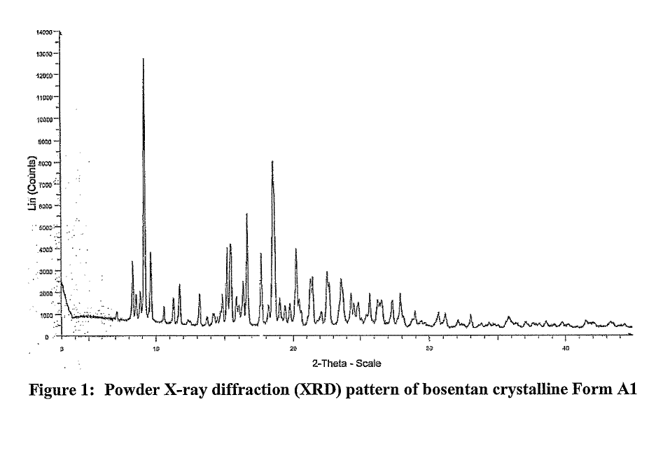

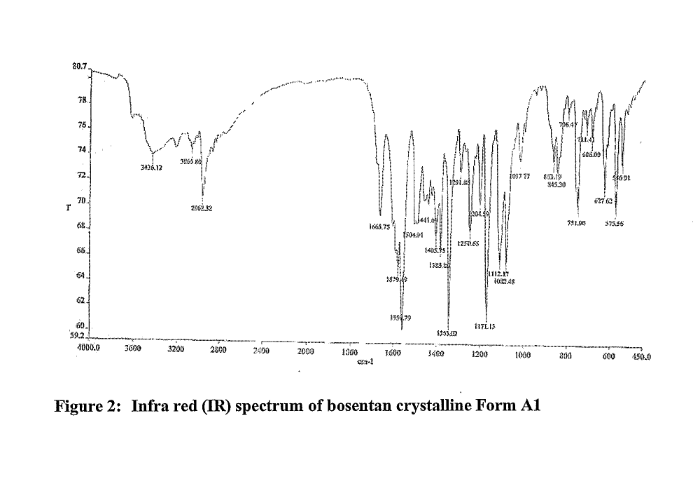

According to one aspect of the present invention, there is provided a novel crystalline form of bosentan, designated as crystalline Form A1, characterized by at least one, and preferably all, of the following properties:
i) a powder X-ray diffraction pattern substantially in accordance with FIG. 1;
ii) a powder X-ray diffraction pattern having peaks at about 9.62, 16.34, 18.18 and 22.08±0.2 degrees 2-theta substantially as depicted in FIG. 1;
iii) a powder X-ray diffraction pattern having additional peaks at about 8.27, 8.52, 8.84, 9.18, 11.26, 11.71, 13.15, 14.81, 15.18, 15.45, 15.84, 16.64, 17.67, 18.58, 19.02, 20.24, 21.39, 22.58, 23.62, 24.32, 24.83, 26.31, 26.57, 27.30 and 27.91±0.2 degrees 2-theta substantially as depicted in FIG. 1;
iv) an IR spectrum substantially in accordance with FIG. 2; and
v) an IR spectrum having absorption bands at about 3436, 1663, 1112, 1017 and 711±1 cm⁻¹ substantially as depicted in FIG. 2.

According to another aspect of the present invention, a process for the preparation of bosentan crystalline Form A1 is provided, which comprises:
a) providing a solution of bosentan in a suitable organic solvent in an amount of less than about 3 ml per gram of bosentan;
b) combining the solution obtained in step-(a) with water; and
c) recovering bosentan substantially in crystalline Form A1.

The process can produce crystalline Form A1 of bosentan in substantially pure form.

The term “substantially pure bosentan crystalline Form A1” refers to the bosentan crystalline Form A1 having purity greater than about 98%, specifically greater than about 99%, more specifically greater than about 99.5% and still more specifically greater than about 99.9% (measured by HPLC).

The bosentan crystalline Form A1 is stable, consistently reproducible and has good flow properties, and which is particularly suitable for bulk preparation and handling, and so, the novel bosentan crystalline Form A1 is suitable for formulating bosentan.

In a preferred embodiment, the crystalline Form A1 of bosentan obtained according the present invention having water content of about 2.5-4% by weight, specifically about 2.5-3.5% by weight, and more specifically about 2.8-3.5% by weight.

The suitable organic solvent used in step-(a) is selected from the group comprising alcohols, ketones, nitriles, cyclic ethers, aliphatic ethers and mixtures thereof. Preferable solvents are ketones, alcohols and mixtures thereof, and most preferably methanol, ethanol, isopropyl alcohol, acetone and mixtures thereof.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof. Exemplary ketone solvents include, but are not limited to, acetone, methyl ethyl ketone, methyl isobutyl ketone, methyl tert-butyl ketone and the like, and mixtures thereof. Exemplary nitrile solvents include, but are not limited to, acetonitrile, propionitrile and the like, and mixtures thereof. Exemplary cyclic ether solvents include, but are not limited to, tetrahydrofuran, dioxane, and the like, and mixtures thereof. Exemplary aliphatic ether solvents include, but are not limited to, diethyl ether, diisopropyl ether, monoglyme, diglyme and the like, and mixtures thereof.

Step-(a) of providing a solution of bosentan includes dissolving bosentan in the organic solvent, or obtaining an existing solution from a previous processing step.

Preferably the bosentan is dissolved in the organic solvent at a temperature of about 0° C. to about the reflux temperature of the solvent used, more preferably at about 30° C. to about 110° C., and still more preferably at about 50° C. to about 100° C.

As used herein, “reflux temperature” means the temperature at which the solvent or solvent system refluxes or boils at atmospheric pressure.

The solution in step-(a) may be prepared by reacting 4-t-butyl-N-[6-chloro-5-(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan followed by usual work up such as washings, extractions etc., and dissolving the resulting crude bosentan in the organic solvent at a temperature of about 0° C. to about the reflux temperature of the solvent used, more preferably at about 30° C. to about 110° C., and still more preferably at about 50° C. to about 100° C.

Alternatively, the solution in step-(a) may be prepared by treating a pharmaceutically acceptable salt of bosentan with an acid to liberate bosentan and dissolving the bosentan in the organic solvent.

Preferable pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ether solvents, alcoholic solvents, ketonic solvents, ester solvents etc., can be used.

The acid can be inorganic or organic. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

Preferably the organic solvent in an amount of about 1 ml to about 3 ml per gram of bosentan is used, more preferably about 1.8 ml to about 2.8 ml per gram of bosentan is used, and most preferably 2 ml per gram of bosentan is used.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. Preferably, the finely powdered carbon is an active carbon.

The combining of the solution with water in step-(b) is done in a suitable order, for example, the solution is added to the water, or alternatively, the water is added to the solution. The addition is carried out drop wise, in one portion, or in more than one portion. In one embodiment, addition is carried out at a temperature of below about 110° C. for at least 15 minutes, and more specifically at a temperature of about 30° C. to about 100° C. from about 20 minutes to about 2 hours. After completion of addition process, the resulting mass is stirred for at least 20 minutes, more specifically about 30 minutes to about 4 hours, at a temperature of about 20° C. to about 30° C.

Usually, about 0.5 to 3.0 volumes, specifically, about 1 to 2 volumes of water with respect to the organic solvent is used.

The term “Anti-solvent” refers to a solvent which when added to an existing solution of a substance reduces the solubility of the substance.

The recovering in step-(c) is carried out by conventional techniques known in the art such as filtration, filtration under vacuum, decantation, and centrifugation, or a combination thereof, and then dried to obtain substantially pure bosentan crystalline Form A1. In one embodiment, bosentan crystalline Form A1 can be isolated by filtration employing a filtration media of, for example, a silica gel or celite.

The pure bosentan crystalline Form A1 obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

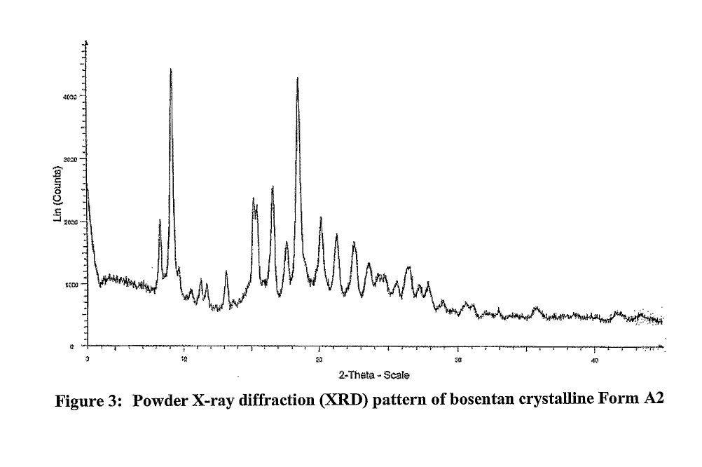

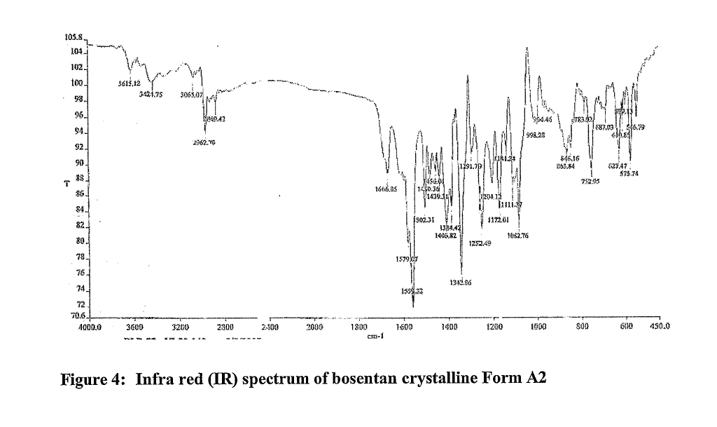

According to another aspect of the present invention, there is provided a novel crystalline form of bosentan, designated as crystalline Form A2, characterized by at least one, and preferably all, of the following properties:
i) a powder X-ray diffraction pattern substantially in accordance with FIG. 3;
ii) a powder X-ray diffraction pattern having peaks at about 8.26, 9.15, 15.21, 15.42, 16.63, 18.55 and 30.39±0.2 degrees 2-theta substantially as depicted in FIG. 3;
iii) a powder X-ray diffraction pattern having additional peaks at about 11.24, 11.69, 13.13, 14.75, 17.63, 19.02, 20.18, 22.58, 23.61, 24.31, 24.74, 26.53 and 27.87±0.2 degrees 2-theta substantially as depicted in FIG. 3;
iv) an IR spectrum substantially in accordance with FIG. 4; and
v) an IR spectrum having absorption bands at about 3615, 3424, 2869, 1666, 1082, 998, 964 and 599±1 cm⁻¹ substantially as depicted in FIG. 4.

According to another aspect of the present invention, a process for the preparation of bosentan crystalline Form A2 is provided, which comprises:

a) providing a solution of bosentan in an aromatic hydrocarbon solvent;
b) combining the solution obtained in step-(a) with an anti-solvent; and
c) recovering bosentan substantially in crystalline Form A2.

The process can produce crystalline Form A2 of bosentan in substantially pure form.

The term “substantially pure bosentan crystalline Form A2” refers to the bosentan crystalline Form A2 having purity greater than about 98%, specifically greater than about 99%, more specifically greater than about 99.5% and still more specifically greater than about 99.9% (measured by HPLC).

The bosentan crystalline Form A2 is stable, consistently reproducible and has good flow properties, and which is particularly suitable for bulk preparation and handling, and so, the novel bosentan crystalline Form A2 is suitable for formulating bosentan.

In a preferred embodiment, the crystalline Form A2 of bosentan obtained according the present invention having water content of about 1.6-2.6% by weight, specifically about 1.8-2.5% by weight, and more specifically about 2.0-2.4% by weight.

Exemplary aromatic hydrocarbon solvents include, but are not limited to, C₆ to C₁₂ aromatic hydrocarbon solvents such as benzene, alkyl substituted benzenes, and mixtures thereof. Specific aromatic hydrocarbon solvents are toluene, xylene, and mixtures thereof, and more specifically toluene.

Step-(a) of providing a solution of bosentan includes dissolving bosentan in the aromatic hydrocarbon solvent, or obtaining an existing solution from a previous processing step.

Preferably the bosentan is dissolved in the aromatic hydrocarbon solvent at a temperature of about 0° C. to about the reflux temperature of the solvent used, more preferably at about 30° C. to about 110° C., and still more preferably at about 50° C. to about 100° C.

As used herein, “reflux temperature” means the temperature at which the solvent or solvent system refluxes or boils at atmospheric pressure.

The solution in step-(a) may be prepared by reacting 4-t-butyl-N-[6-chloro-5-(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan followed by usual work up such as washings, extractions etc., and dissolving the resulting crude bosentan in the aromatic hydrocarbon solvent at a temperature of about 0° C. to about the reflux temperature of the solvent used, more preferably at about 30° C. to about 110° C., and still more preferably at about 50° C. to about 100° C.

Alternatively, the solution in step-(a) may be prepared by treating a pharmaceutically acceptable salt of bosentan with an acid to liberate bosentan and dissolving the bosentan in the aromatic hydrocarbon solvent.

Preferable pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ether solvents, alcoholic solvents, ketonic solvents, ester solvents etc., can be used.

The acid can be inorganic or organic. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. Preferably, the finely powdered carbon is an active carbon.

The anti-solvent used in step-(b) include, but are not limited to, C₃ to C₇ straight or cyclic aliphatic hydrocarbon solvents such as hexane, heptane, cyclopentane, cyclohexane, cycloheptane, and mixtures thereof; and ether solvents such as diisopropyl ether, diethyl ether, tetrahydrofuran, dioxane, and the like, and mixtures thereof. Specific anti-solvents are hexane, heptane, cyclohexane, and mixtures thereof, and more specifically hexane.

The term “Anti-solvent” refers to a solvent which when added to an existing solution of a substance reduces the solubility of the substance.

The combining of the solution with anti-solvent in step-(b) is done in a suitable order, for example, the solution is added to the anti-solvent, or alternatively, the anti-solvent is added to the solution. The addition is carried out drop wise, in one portion, or in more than one portion. In one embodiment, addition is carried out at a temperature of below about 110° C. for at least 15 minutes, and more specifically at a temperature of about 30° C. to about 100° C. from about 20 minutes to about 2 hours. After completion of addition process, the resulting mass is stirred for at least 20 minutes, more specifically about 30 minutes to about 4 hours, at a temperature of about 20° C. to about 30° C.

Usually, about 1 to 6 volumes, specifically, about 2 to 5 volumes of anti-solvent with respect to the aromatic hydrocarbon solvent is used.

The term “Anti-solvent” refers to a solvent which when added to an existing solution of a substance reduces the solubility of the substance.

The recovering in step-(c) is carried out by conventional techniques known in the art such as filtration, filtration under vacuum, decantation, and centrifugation, or a combination thereof, and then dried to obtain substantially pure bosentan crystalline Form A2. In one embodiment, bosentan crystalline Form A2 can be isolated by filtration employing a filtration media of, for example, a silica gel or celite.

The pure bosentan crystalline Form A2 obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

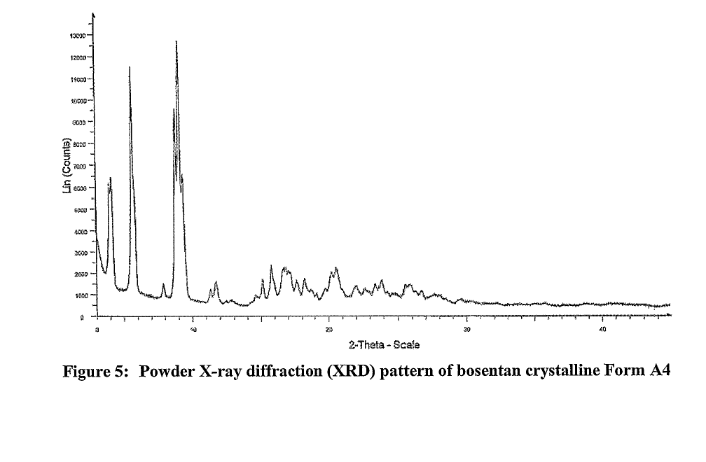

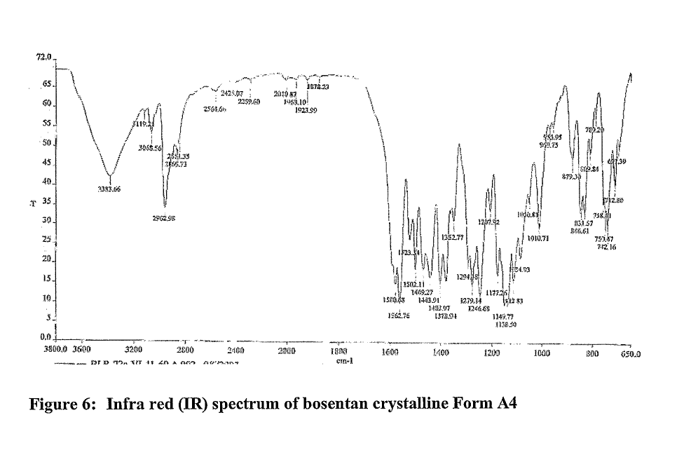

According to another aspect of the present invention, there is provided a novel crystalline form of bosentan, designated as crystalline Form A4, characterized by at least one, and preferably all, of the following properties:
i) a powder X-ray diffraction pattern substantially in accordance with FIG. 5;
ii) a powder X-ray diffraction pattern having peaks at about 4.04, 5.62, 7.84 and 17.06±0.2 degrees 2-theta substantially as depicted in FIG. 5;
iii) a powder X-ray diffraction pattern having additional peaks at about 8.79, 9.03, 9.30, 11.67, 15.10, 15.76, 16.71, 18.19, 20.21 and 20.56±0.2 degrees 2-theta substantially as depicted in FIG. 5;
iv) an IR spectrum substantially in accordance with FIG. 6; and
v) an IR spectrum having absorption bands at about 3383, 3068, 1443, 1378, 1352, 1246, 1207, 1177, 1138, 1050, 1010, 969, 831, 742 and 697±1 cm⁻¹ substantially as depicted in FIG. 6.

According to another aspect of the present invention, a process for the preparation of bosentan crystalline Form A4 is provided, which comprises:

a) providing a solution of bosentan in a suitable organic solvent;
b) optionally, filtering the solvent solution to remove any extraneous matter; and
c) isolating bosentan substantially in crystalline Form A4.

The process can produce crystalline Form A4 of bosentan in substantially pure form.

The term “substantially pure bosentan crystalline Form A4” refers to the bosentan crystalline Form A4 having purity greater than about 98%, specifically greater than about 99%, more specifically greater than about 99.5% and still more specifically greater than about 99.9% (measured by HPLC).

The bosentan crystalline Form A4 is stable, consistently reproducible and has good flow properties, and which is particularly suitable for bulk preparation and handling, and so, the novel bosentan crystalline Form A4 is suitable for formulating bosentan.

In a preferred embodiment, the crystalline Form A4 of bosentan obtained according the present invention having water content of about 0.5-1.5% by weight, specifically about 0.8-1.5% by weight, and more specifically about 1.0-1.5% by weight.

The suitable organic solvent used in step-(a) is selected from the group comprising alcohols, ketones, nitriles, cyclic ethers, aliphatic ethers and mixtures thereof. Preferable solvents are ketones, alcohols and mixtures thereof, and most preferably methanol, ethanol, isopropyl alcohol, acetone and mixtures thereof.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof. Exemplary ketone solvents include, but are not limited to, acetone, methyl ethyl ketone, methyl isobutyl ketone, methyl tert-butyl ketone and the like, and mixtures thereof. Exemplary nitrile solvents include, but are not limited to, acetonitrile, propionitrile and the like, and mixtures thereof. Exemplary cyclic ether solvents include, but are not limited to, tetrahydrofuran, dioxane, and the like, and mixtures thereof. Exemplary aliphatic ether solvents include, but are not limited to, diethyl ether, diisopropyl ether, monoglyme, diglyme and the like, and mixtures thereof.

Step-(a) of providing a solution of bosentan includes dissolving bosentan in the organic solvent, or obtaining an existing solution from a previous processing step.

Preferably the bosentan is dissolved in the organic solvent at a temperature of about 0° C. to about the reflux temperature of the solvent used, more preferably at about 30° C. to about 110° C., and still more preferably at about 50° C. to about 100° C.

As used herein, “reflux temperature” means the temperature at which the solvent or solvent system refluxes or boils at atmospheric pressure.

The solution in step-(a) may be prepared by reacting 4-t-butyl-N-[6-chloro-5-(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan followed by usual work up such as washings, extractions etc., and dissolving the resulting crude bosentan in the organic solvent at a temperature of about 0° C. to about the reflux temperature of the solvent used, more preferably at about 30° C. to about 110° C., and still more preferably at about 50° C. to about 100° C.

Alternatively, the solution in step-(a) may be prepared by treating a pharmaceutically acceptable salt of bosentan with an acid to liberate bosentan and dissolving the bosentan in the organic solvent.

Preferable pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ether solvents, alcoholic solvents, ketonic solvents, ester solvents etc., can be used.

The acid can be inorganic or organic. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

Preferably the organic solvent in an amount of about 3.5 ml to about 5.5 ml per gram of bosentan is used, more preferably about 4.0 ml to about 5.2 ml per gram of bosentan is used, and most preferably about 4.5 ml to about 5.0 ml per gram of bosentan is used.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. Preferably, the finely powdered carbon is an active carbon.

The solution obtained in step-(a) is preferably heated at a temperature of about 40° C. to about 90° C. for at least 20 minutes, and more preferably at a temperature of about 40° C. to about 80° C. from about 30 minutes to about 4 hours.

The isolation of pure bosentan crystalline Form A4 in step-(c) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

Preferably the crystallization is carried out by cooling the solution at a temperature of below about 30° C. for at least 15 minutes, more preferably at about 0° C. to about 25° C. from about 30 minutes to about 10 hours, and still more preferably at about 15° C. to about 25° C. from about 1 hour to about 5 hours.

The solid obtained in step-(c) is collected by conventional techniques known in the art such as filtration, filtration under vacuum, decantation, centrifugation, filtration employing a filtration media selected from silica gel and celite, or a combination thereof.

The pure bosentan crystalline Form A4 obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

According to another aspect of the present invention, there is provided a stable amorphous form of bosentan.

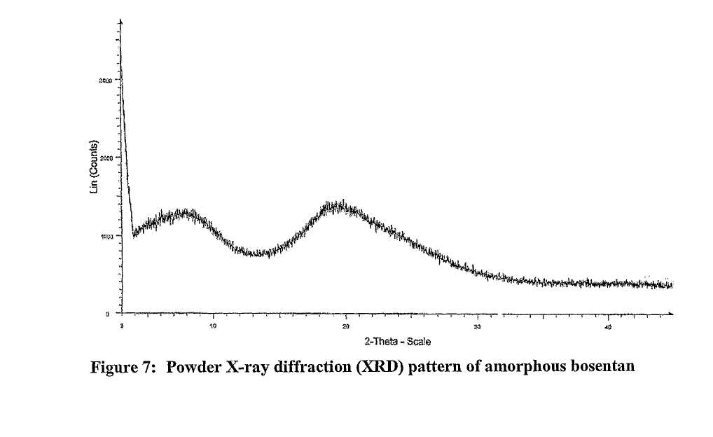

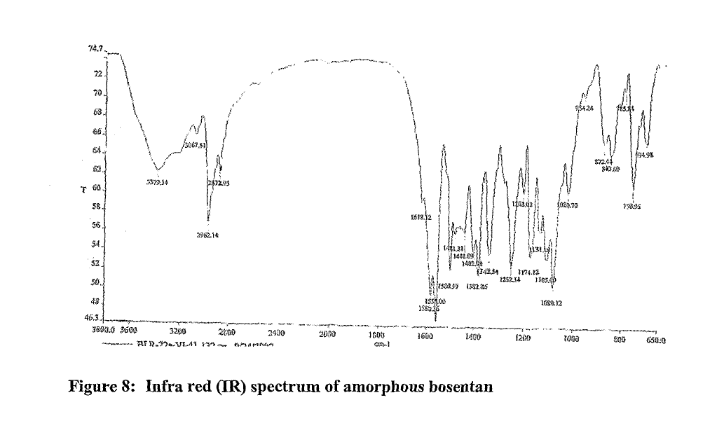

Amorphous form of bosentan is characterized by at least one, and preferably all, of the following properties: a powder XRD pattern substantially in accordance with FIG. 7; an IR spectrum substantially in accordance with FIG. 8; and an IR spectrum having absorption bands at about 3379, 3067, 2872, 1618, 1500, 1441, 1382, 1174, 1131, 1080, 1020, 843 and 694±1 cm⁻¹ substantially as depicted in FIG. 8. The X-ray powder diffraction pattern shows no peaks, thus demonstrating the amorphous nature of the product.

According to another aspect of the present invention, a process is provided for preparation of amorphous form of bosentan, which comprises:
a) providing a solution of bosentan in a suitable solvent or a mixture of solvents capable of dissolving bosentan;
b) optionally, filtering the solvent solution to remove any extraneous matter; and
c) substantially removing the solvent from the solution to afford amorphous form of bosentan.

The process can produce amorphous bosentan in substantially pure form.

The term “substantially pure amorphous form of bosentan” refers to the amorphous form of bosentan having purity greater than about 98%, specifically greater than about 99%, more specifically greater than about 99.5% and still more specifically greater than about 99.9% (measured by HPLC).

In a preferred embodiment, the amorphous form of bosentan obtained according the present invention having water content less than about 4% by weight, specifically less than about 3.5% by weight, and more specifically less than about 1% by weight, and still more specifically is essentially free from water.

The amorphous bosentan obtained by the process disclosed herein is stable, consistently reproducible and has good dissolution properties, and which is particularly suitable for bulk preparation and handling, and so, the amorphous bosentan obtained by the process disclosed herein is suitable for formulating bosentan.

The suitable solvent used in step-(a) is selected from the group comprising water, alcohols, ketones, chlorinated hydrocarbons, nitriles, esters, cyclic ethers, aliphatic ethers, polar aprotic solvents, and mixtures thereof. Preferable solvents are chlorinated hydrocarbons, ketones, alcohols and mixtures thereof, more preferably ketones, alcohols and mixtures thereof, and most preferably methanol, ethanol, isopropyl alcohol, acetone and mixtures thereof.

Exemplary alcohol solvents include, but are not limited to, Cᵢ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof, and most specific alcohol solvent is methanol. Exemplary ketone solvents include, but are not limited to, acetone, methyl ethyl ketone, methyl isobutyl ketone, methyl tert-butyl ketone and the like, and mixtures thereof. Exemplary nitrile solvents include, but are not limited to, acetonitrile, propionitrile and the like, and mixtures thereof. Exemplary ester solvents include, but are not limited to, ethyl acetate, isopropyl acetate, and the like and mixtures thereof. Exemplary chlorinated hydrocarbon solvents include, but are not limited to, methylene chloride, ethyl dichloride, chloroform, carbon tetrachloride, and mixtures thereof. Specific chlorinated hydrocarbon solvent is methylene chloride. Exemplary cyclic ether solvents include, but are not limited to, tetrahydrofuran, dioxane, and the like, and mixtures thereof. Exemplary aliphatic ether solvents include, but are not limited to, diethyl ether, diisopropyl ether, monoglyme, diglyme and the like, and mixtures thereof. Exemplary polar aprotic solvents include, but are not limited to, N,N-dimethylformamide, N,N-dimethylacetamide, dimethylsulfoxide, and mixtures thereof.

Step-(a) of providing a solution of bosentan includes dissolving bosentan in the solvent, or obtaining an existing solution from a previous processing step.

Preferably the bosentan is dissolved in the solvent at a temperature of about 0° C. to about the reflux temperature of the solvent used, more preferably at about 25° C. to about 110° C., and still more preferably at about 30° C. to about 90° C.

As used herein, “reflux temperature” means the temperature at which the solvent or solvent system refluxes or boils at atmospheric pressure.

The solution in step-(a) may be prepared by reacting 4-t-butyl-N-[6-chloro-5-(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan followed by usual work up such as washings, extractions etc., and dissolving the resulting crude bosentan in the solvent at a temperature of about 0° C. to about the reflux temperature of the solvent used, more preferably at about 25° C. to about 110° C., and still more preferably at about 30° C. to about 90° C.

Alternatively, the solution in step-(a) may be prepared by treating a pharmaceutically acceptable salt of bosentan with an acid to liberate bosentan and dissolving the bosentan in the solvent.

Preferable pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ether solvents, alcoholic solvents, ketonic solvents, ester solvents etc., can be used.

The acid can be inorganic or organic. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. Preferably, the finely powdered carbon is an active carbon.

The solution obtained in step-(a) is preferably heated at a temperature of about 30° C. to about 90° C. for at least 20 minutes, and more preferably at a temperature of about 35° C. to about 80° C. from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The pure amorphous bosentan obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

Bosentan used as starting material can be obtained by processes described in the prior art, for example by the process described in the U.S. Pat. No. 5,292,740.

Karl Fisher analysis, which is well known in the art, is also used to determine the quantity of water in a sample.

In one embodiment, any one or a mixture of the substantially pure polymorphic forms of bosentan (Form A1, Form A2, Form A4 and amorphous form) disclosed herein is used in pharmaceutical compositions, wherein 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure polymorphic forms of bosentan is achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect, there is provided pharmaceutical compositions comprising a therapeutically effective amount of each one of bosentan polymorphic forms disclosed herein and one or more pharmaceutically acceptable excipients.

In another embodiment, provided herein is a pharmaceutical composition comprising a therapeutically effective amount of any one or a mixture of the polymorphic forms of bosentan disclosed herein, and one or more pharmaceutically acceptable excipients.

According to another aspect, there are provided pharmaceutical compositions comprising the polymorphic forms of bosentan prepared according to processes disclosed herein and one or more pharmaceutically acceptable excipients.

According to another aspect, there is provided a process for preparing a pharmaceutical formulation comprising combining any one or a mixture of the polymorphic forms of bosentan prepared according to processes disclosed herein, with one or more pharmaceutically acceptable excipients.

Yet another embodiment is directed to pharmaceutical compositions comprising at least a therapeutically effective amount of any one of the substantially pure polymorphic forms of bosentan disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The polymorphic forms of bosentan may also be administered as suppositories, ophthalmic ointments and suspensions, and parenteral suspensions, which are administered by other routes. The dosage forms may contain any one of the polymorphic forms of bosentan as is or, alternatively, may contain any one of the polymorphic forms of bosentan of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In another embodiment of the present invention, there is provided a pharmaceutical composition comprising bosentan crystalline Form A1 and one or more pharmaceutically acceptable excipients.

In another embodiment of the present invention, there is provided a pharmaceutical composition comprising bosentan crystalline Form A2 and one or more pharmaceutically acceptable excipients.

In another embodiment of the present invention, there is provided a pharmaceutical composition comprising bosentan crystalline Form A4 and one or more pharmaceutically acceptable excipients.

In another embodiment of the present invention, there is provided a pharmaceutical composition comprising amorphous form of bosentan and one or more pharmaceutically acceptable excipients.

Capsule dosages, for example, contain the polymorphic forms of bosentan within a capsule which may be coated with gelatin. Tablets and powders are optionally coated with an enteric coating. The enteric-coated powder form have coatings containing, for example, phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are provided to enable one skilled in the art to practice the invention and are merely illustrate the process of this invention. However, it is not intended in any way to limit the scope of the present invention.

In the following examples, the products had the spectral properties of the named products as shown in the Figures. The crystalline forms show good stability suitable for use as pharmaceutical agents. The amorphous form shows advantageous dissolution properties suitable for use as a pharmaceutical agent.

### EXAMPLES

### Example 1

### Preparation of Crystalline Form A1 of Bosentan

Bosentan (10 g) was taken in ethanol (20 ml) and heated at 70 to 80° C. for 10-15 minutes to get the clear solution. This was followed by the addition of water (20 ml) at 70 to 80° C. and then further gradually cooled at 20 to 25° C. The reaction mixture was then stirred for 3 hours at 20 to 25° C. The resulted solid was filtered and washed with 1:1 mixture of ethanol (2.5 ml) and water (2.5 ml). The resulted solid was dried at 60-65° C. to give 9.6 g of bosentan in crystalline Form A1 (Moisture content: 3.02% w/w).

### Example 2

### Preparation of Crystalline Form A1 of Bosentan

Bosentan (10 g) was taken in acetone (20 ml) and heated at 60 to 65° C. for 10-15 minutes. This was followed by the addition of water (20 ml) at 60 to 65° C. The reaction mixture was then cooled at 20 to 25° C. The reaction mixture was further stirred for 3 hours at 20 to 25° C. The resulted solid was filtered and washed with 1:1 mixture of acetone (2.5 ml) and water (2.5 ml). The resulted solid was dried at 60-65° C. to yield 9.6 g of bosentan in crystalline Form A1 (Moisture content: 3.04% w/w).

### Example 3

### Preparation of Crystalline Form A2 of Bosentan

Bosentan (5 g) was taken in toluene (60 ml) and heated at 75° C. for 10-15 minutes. The resulted mass was stirred for 10 minutes and filtered to get the clear solution. This was followed by the addition of hexane (180 ml) at 75° C. and then further gradually cooled at 20 to 25° C. The reaction mass stirred for 1 hour at 20 to 25° C. The resulted solid was filtered and washed with hexane (10 ml). The obtained solid was dried at 60-65° C. to yield 3.4 g of bosentan in crystalline Form A2 (Moisture content: 2.13% w/w).

### Example 4

### Preparation of Crystalline Form A4 of Bosentan

Bosentan (20 g) was dissolved methanol (85 ml) and heated at 60 to 65° C. for 15 minutes and filtered through hyflo to remove insoluble solids. The hyflo bed was washed with methanol (5 ml) and the resulting filtrate was stirred for 2 hours at 20 to 30° C. The resulted mass was dried at 60 to 65° C. till moisture content is 1 to 1.5% to yield 6.4 g of bosentan crystalline Form A4.

### Example 5

### Preparation of Crystalline Form A4 of Bosentan

Bosentan (10 g) was taken in ethanol (40 ml) and heated at 60 to 65° C. for 15 minutes and filtered through hyflo to remove insoluble solids. The hyflo bed was washed with ethanol (10 ml) and resulted filtrate was stirred for 12 hours at 20 to 30° C. The resulted mass was filtered and then dried at 60 to 65° C. till moisture content is 1 to 1.5% to yield 8.2 g of bosentan in crystalline Form A4.

### Example 6

### Preparation of Amorphous Bosentan

Bosentan (1 g) was taken in methanol (20 ml) and heated at 65° C. for 10-15 minutes. The reaction mass was filtered through hyflo bed to get clear solution and washed the bed with methanol (10 ml). Solvent was removed by drying under vacuum using rotary evaporator at 50 to 55° C. The resulted solid was collected to give 0.95 g of bosentan in amorphous form.

### Example 7

### Preparation of Amorphous Bosentan

Bosentan (1 g) was taken in acetonitrile (20 ml) and heated at 35° C. The reaction mass was filtered through hyflo bed to get clear solution and washed the bed with acetonitrile (10 ml). Acetonitrile was distilled off under vacuum using rotary evaporator at 50 to 55° C. to give 0.95 g of bosentan in amorphous form.

### Example 8

### Preparation of Amorphous Bosentan

Bosentan (1 g) was taken in dichloromethane (20 ml) and heated at 40° C. The reaction mass was filtered through hyflo bed to get the clear solution. Dichloromethane was removed under vacuum using rotary evaporator at 50 to 55° C. to give 0.85 g of bosentan in amorphous form.

We claim:

1. Bosentan characterized as being more than 98% pure:
a) in the crystalline Form A1;
b) in the crystalline Form A2;
c) in the crystalline Form A4;
d) in amorphous form;
wherein:
e) the crystalline Form A1 has at least one of the following characteristics:
i) a powder X-ray diffraction pattern substantially in accordance with FIG. 1;
ii) a powder X-ray diffraction pattern having peaks at about 9.62, 16.34, 18.18 and 22.08±0.2 degrees 2-theta substantially as depicted in FIG. 1;
iii) a powder X-ray diffraction pattern having additional peaks at about 8.27, 8.52, 8.84, 9.18, 11.26, 11.71, 13.15, 14.81, 15.18, 15.45, 15.84, 16.64, 17.67, 18.58, 19.02, 20.24, 21.39, 22.58, 23.62, 24.32, 24.83, 26.31, 26.57, 27.30 and 27.91±0.2 degrees 2-theta substantially as depicted in FIG. 1;
iv) an IR spectrum substantially in accordance with FIG. 2; and
v) an IR spectrum having absorption bands at about 3436, 1663, 1112, 1017 and 711±1 cm⁻¹ substantially as depicted in FIG. 2;
f) the crystalline Form A2 has at least one of the following characteristics:
i) a powder X-ray diffraction pattern substantially in accordance with FIG. 3;
ii) a powder X-ray diffraction pattern having peaks at about 8.26, 9.15, 15.21, 15.42, 16.63, 18.55 and 30.39±0.2 degrees 2-theta substantially as depicted in FIG. 3;
iii) a powder X-ray diffraction pattern having additional peaks at about 11.24, 11.69, 13.13, 14.75, 17.63, 19.02, 20.18, 22.58, 23.61, 24.31, 24.74, 26.53 and 27.87±0.2 degrees 2-theta substantially as depicted in FIG. 3;
iv) an IR spectrum substantially in accordance with FIG. 4; and
v) an IR spectrum having absorption bands at about 3615, 3424, 2869, 1666, 1082, 998, 964 and 599±1 cm⁻¹ substantially as depicted in FIG. 4;
g) the crystalline Form A4 has at least one of the following characteristics:
i) a powder X-ray diffraction pattern substantially in accordance with FIG. 5;
ii) a powder X-ray diffraction pattern having peaks at about 4.04, 5.62, 7.84 and 17.06±0.2 degrees 2-theta substantially as depicted in FIG. 5;
iii) a powder X-ray diffraction pattern having additional peaks at about 8.79, 9.03, 9.30, 11.67, 15.10, 15.76, 16.71, 18.19, 20.21 and 20.56±0.2 degrees 2-theta substantially as depicted in FIG. 5;
iv) an IR spectrum substantially in accordance with FIG. 6; and
v) an IR spectrum having absorption bands at about 3383, 3068, 1443, 1378, 1352, 1246, 1207, 1177, 1138, 1050, 1010, 969, 831, 742 and 697±1 cm⁻¹ substantially as depicted in FIG. 6;
h) the amorphous Form has at least one of the following characteristics:
i) a powder XRD pattern substantially in accordance with FIG. 7;
ii) an IR spectrum substantially in accordance with FIG. 8; and
iii) an IR spectrum having absorption bands at about 3379, 3067, 2872, 1618, 1500, 1441, 1382, 1174, 1131, 1080, 1020, 843 and 694±1 cm⁻¹ substantially as depicted in FIG. 8.

2. A crystalline Form A1 of bosentan characterized by at least one, or more, of the following properties:
i) a powder X-ray diffraction pattern substantially in accordance with FIG. 1;
ii) a powder X-ray diffraction pattern having peaks at about 9.62, 16.34, 18.18 and 22.08±0.2 degrees 2-theta substantially as depicted in FIG. 1;
iii) a powder X-ray diffraction pattern having additional peaks at about 8.27, 8.52, 8.84, 9.18, 11.26, 11.71, 13.15, 14.81, 15.18, 15.45, 15.84, 16.64, 17.67, 18.58, 19.02, 20.24, 21.39, 22.58, 23.62, 24.32, 24.83, 26.31, 26.57, 27.30 and 27.91±0.2 degrees 2-theta substantially as depicted in FIG. 1;
iv) an IR spectrum substantially in accordance with FIG. 2; and
v) an IR spectrum having absorption bands at about 3436, 1663, 1112, 1017 and 711±1 cm⁻¹ substantially as depicted in FIG. 2.

3. A process for the preparation of bosentan crystalline Form A1 of claim 2, comprising:
a) providing a solution of bosentan in an organic solvent in an amount of less than about 3 ml per gram of bosentan, wherein the organic solvent used in step-(a) is selected from the group consisting of alcohols, ketones, nitriles, cyclic ethers, aliphatic ethers and mixtures thereof;
b) combining the solution obtained in step-(a) with water; and
c) recovering bosentan substantially in crystalline Form A1, wherein the recovering is carried out by filtration, filtration under vacuum, decantation, centrifugation, filtration employing a filtration media selected from silica gel and celite, or a combination thereof.

4. The process of claim 3, wherein the crystalline Form A1 of bosentan obtained has water content of about 2.5-4% by weight; wherein the organic solvent used in step-(a) is selected from the group consisting of methanol, ethanol, n-propanol, isopropyl alcohol, n-butanol, tert-butanol, amyl alcohol, hexanol, acetone, methyl ethyl ketone, methyl isobutyl ketone, methyl tert-butyl ketone, acetonitrile, propionitrile, tetrahydrofuran, dioxane, diethyl ether, diisopropyl ether, monoglyme, diglyme, and mixtures thereof; wherein the organic solvent in step-(a) is used in an amount of about 1 ml to about 3 ml per gram of bosentan; wherein the solution obtained in step-(a) is optionally subjected to carbon treatment; wherein the combining in step-(b) is carried out by adding water to the bosentan solution or by adding the bosentan solution to water; and wherein the bosentan crystalline Form A1 obtained in step-(c) is further dried under atmospheric pressure or reduced pressures at a temperature of about 35° C. to about 70° C.

5. The process of claim 4, wherein the crystalline Form A1 of bosentan has water content of about 2.8-3.5% by weight; wherein the organic solvent used in step-(a) is selected from the group consisting of methanol, ethanol, isopropyl alcohol, acetone and mixtures thereof; wherein the organic solvent in step-(a) is used in an amount of about 1.8 ml to about 2.8 ml per gram of bosentan; wherein the addition in step-(b) is carried out at a temperature of about 30° C. to about 100° C. from about 20 minutes to about 2 hours; and wherein the reaction mass obtained after addition of water in step-(b) is further stirred for at least 20 minutes at a temperature of about 20° C. to about 30° C.

6-22. (canceled)

23. A hydrated crystalline Form A2 of bosentan having water content of about 1.6 to about 2.6% by weight, characterized by at least one, or more, of the following properties:
i) a powder X-ray diffraction pattern substantially in accordance with FIG. 3.
ii) a powder X-ray diffraction pattern having peaks at about 8.26, 9.15, 15.21, 15.42, 16.63, 18.55 and 30.39±0.2 degrees 2-theta substantially as depicted in FIG. 3;
iii) a powder X-ray diffraction pattern having additional peaks at about 11.24, 11.69, 13.13, 14.75, 17.63, 19.02, 20.18, 22.58, 23.61, 24.31, 24.74, 26.53 and 27.87±0.2 degrees 2-theta substantially as depicted in FIG. 3;
iv) an IR spectrum substantially in accordance with FIG. 4; and
v) an IR spectrum having absorption bands at about 3615, 3424, 2869, 1666, 1082, 998, 964 and 599±1 cm⁻¹ substantially as depicted in FIG. 4.

24. (canceled)

25. A process for the preparation of bosentan crystalline Form A2 of claim 23, comprising:
a) providing a solution of bosentan in an aromatic hydrocarbon solvent;
b) combining the solution obtained in step-(a) with an anti-solvent, wherein the anti-solvent is selected from the group consisting of C₃ to C₇ straight or cyclic aliphatic hydrocarbon solvents, ether solvents, and mixtures thereof; and
c) recovering bosentan substantially in crystalline Form A2, wherein the recovering is carried out by filtration, filtration under vacuum, decantation, centrifugation, filtration employing a filtration media selected from silica gel and celite, or a combination thereof.

26. The process of claim 25, wherein the crystalline Form A2 of bosentan obtained has water content of about 1.8-2.5% by weight; wherein the aromatic hydrocarbon solvent is selected from the group consisting of benzene, toluene, xylene, and mixtures thereof; wherein the solution obtained in step-(a) is optionally subjected to carbon treatment; wherein the anti-solvent used in step-(b) is selected from the group consisting of hexane, heptane, cyclopentane, cyclohexane, cycloheptane, diisopropyl ether, diethyl ether, tetrahydrofuran, dioxane, and mixtures thereof; wherein the combining in step-(b) is carried out by adding the anti-solvent to the bosentan solution or by adding the bosentan solution to the anti-solvent; and wherein the bosentan crystalline Form A2 obtained in step-(c) is further dried under atmospheric pressure or reduced pressures at a temperature of about 35° C. to about 70° C.

27. The process of claim 26, wherein the crystalline Form A2 of bosentan has water content of about 2.0-2.4% by weight; wherein the aromatic hydrocarbon solvent is toluene; wherein the anti-solvent is hexane; wherein the addition in step-(b) is carried out at a temperature of about 30° C. to about 100° C. for about 20 minutes to about 2 hours; and wherein the reaction mass obtained after addition of anti-solvent in step-(b) is further stirred for at least 20 minutes at a temperature of about 20° C. to about 30° C.

28.-44. (canceled)

45. A hydrated crystalline Form A4 of bosentan having water content of about 0.5 to about 1.5% by weight, characterized by at least one, or more, of the following properties:
i) a powder X-ray diffraction pattern substantially in accordance with FIG. 5.
ii) a powder X-ray diffraction pattern having peaks at about 4.04, 5.62, 7.84 and 17.06±0.2 degrees 2-theta substantially as depicted in FIG. 5;
iii) a powder X-ray diffraction pattern having additional peaks at about 8.79, 9.03, 9.30, 11.67, 15.10, 15.76, 16.71, 18.19, 20.21 and 20.56±0.2 degrees 2-theta substantially as depicted in FIG. 5;
iv) an IR spectrum substantially in accordance with FIG. 6; and
v) an IR spectrum having absorption bands at about 3383, 3068, 1443, 1378, 1352, 1246, 1207, 1177, 1138, 1050, 1010, 969, 831, 742 and 697±1 cm⁻¹ substantially as depicted in FIG. 6.

46. (canceled)

47. A process for the preparation of bosentan crystalline Form A4 of claim 45, comprising:
a) providing a solution of bosentan in an organic solvent selected from the group consisting of alcohols, ketones, and mixtures thereof;
b) heating the solution obtained in step-(a) at a temperature of about 40° C. to about 90° C.;
c) optionally, filtering the solvent solution to remove any extraneous matter; and
d) isolating bosentan substantially in crystalline Form A4 from the solution obtained in step-(b) or step-(c) by cooling the solution while stirring at a temperature of below about 30° C.

48. The process of claim 47, wherein the crystalline Form A4 of bosentan obtained has water content of about 0.8-1.5% by weight; wherein the organic solvent used in step-(a) is selected from the group consisting of methanol, ethanol, n-propanol, isopropyl alcohol, n-butanol, tert-butanol, amyl alcohol, hexanol, acetone, methyl ethyl ketone, methyl isobutyl ketone, methyl tert-butyl ketone, and mixtures thereof; wherein the solution obtained in step-(a) is optionally subjected to carbon treatment; wherein the solution in step-(b) is heated at a temperature of about 40° C. to about 80° C. for about 30 minutes to about 4 hours; wherein the isolation in step-(d) is carried out by cooling the solution at a temperature of about 0° C. to about 25° C. for about 30 minutes to about 10 hours; wherein the solid obtained in step-(d) is collected by filtration, filtration under vacuum, decantation, centrifugation, filtration employing a filtration media selected from silica gel and celite, or a combination thereof.

49. The process of claim 48, wherein the crystalline Form A4 of bosentan has water content of about 1.0-1.5% by weight; wherein the organic solvent used in step-(a) is selected from the group consisting of methanol, ethanol, isopropyl alcohol, acetone and mixtures thereof wherein the isolation in step-(d) is carried out by cooling the solution at a temperature of about 15° C. to about 25° C. for about 1 hour to about 5 hours; and wherein the bosentan crystalline Form A4 obtained in step-(d) is further dried under atmospheric pressure or reduced pressure at a temperature of about 35° C. to about 70° C.

50.-67. (canceled)

68. Amorphous form of bosentan characterized by at least one or more of the following properties:
i) a powder XRD pattern substantially in accordance with FIG. 7;
ii) an IR spectrum substantially in accordance with FIG. 8; and
iii) an IR spectrum having absorption bands at about 3379, 3067, 2872, 1618, 1500, 1441, 1382, 1174, 1131, 1080, 1020, 843 and 694±1 cm⁻¹ substantially as depicted in FIG. 8.

69. A process for the preparation of amorphous bosentan of claim 68, comprising:
a) providing a solution of bosentan in a suitable solvent or a mixture of solvents capable of dissolving bosentan, wherein the solvent or the solvent mixture is selected from the group consisting of water, alcohols, ketones, chlorinated hydrocarbons, nitriles, esters, cyclic ethers, aliphatic ethers, polar aprotic solvents, and mixtures thereof;
b) optionally, filtering the solvent solution to remove any extraneous matter; and
c) substantially removing the solvent from the solution to afford amorphous form of bosentan, wherein the removal of the solvent is accomplished by complete evaporation of the solvent, spray drying, vacuum drying, lyophilization or freeze drying, or a combination thereof.

70. (canceled)

71. The process of claim 69, wherein the solvent or the solvent mixture used in step-(a) is selected from the group consisting of water, methanol, ethanol, n-propanol, isopropyl alcohol, n-butanol, tert-butanol, amyl alcohol, hexanol, acetone, methyl ethyl ketone, methyl isobutyl ketone, methyl tert-butyl ketone, acetonitrile, propionitrile, ethyl acetate, isopropyl acetate, methylene chloride, ethyl dichloride, chloroform, carbon tetrachloride, tetrahydrofuran, dioxane, diethyl ether, diisopropyl ether, monoglyme, diglyme, N,N-dimethylformamide, N,N-dimethylacetamide, dimethylsulfoxide, and mixtures thereof; wherein the solution obtained in step-(a) is optionally subjected to carbon treatment; wherein the solution obtained in step-(a) is heated at a temperature of about 30° C. to about 90° C. for at least 20 minutes; and wherein the amorphous bosentan obtained in step-(c) is further dried under atmospheric pressure or reduced pressure at a temperature of about 35° C. to about 70° C.

72. The process of claim 71, wherein the solvent used in step-(a) is selected from the group consisting of methanol, ethanol, isopropyl alcohol, acetone and mixtures thereof; and wherein the solution obtained in step-(a) is heated at a temperature of about 35° C. to about 80° C. from about 30 minutes to about 4 hours.

73. The process of any one of claims 3, 25, 47 and 69, wherein the solution in step-(a) is provided either i) by dissolving bosentan in the solvent at a temperature of about 0° C. to about the reflux temperature of the solvent used; or ii) by reacting 4-t-butyl-N-[6-chloro-5-(2-methoxyphenoxy)-2-(2-pyrimidinyl)-4-pyrimidinyl]benzene sulfonamide with ethylene glycol in the presence of a suitable base, optionally in the presence of a phase transfer catalyst, in a suitable solvent under suitable conditions to produce a reaction mass containing crude bosentan; subjecting the reaction mass to washings, evaporations or extractions; and dissolving the resulting crude bosentan in the organic solvent at a temperature of about 0° C. to about the reflux temperature of the solvent used or iii) by treating a pharmaceutically acceptable salt of bosentan with an acid to liberate bosentan and dissolving the bosentan in the solvent.

74.-82. (canceled)

83. A pharmaceutical composition comprising a therapeutically effective amount of any one or a mixture of the bosentan polymorphic forms selected from Form A1, Form A2, Form A4 and amorphous form, and one or more pharmaceutically acceptable excipients, wherein the pharmaceutical composition is prepared by a process comprising combining the bosentan polymorphic form of any one of claims 2, 23, 45 and 68, with one or more pharmaceutically acceptable excipients.

84.-89. (canceled)

90. The pharmaceutical composition of claim 83, wherein the polymorphic form of bosentan has a D₉₀ particle size of less than or equal to about 500 microns.

91. The pharmaceutical composition of claim 90, wherein the polymorphic form of bosentan has a D₉₀ particle size of less than or equal to about 300 microns; less than or equal to about 100 microns; less than or equal to about 60 microns; or less than or equal to about 15 microns.

92.-94. (canceled)
